(19) United States
(12) Patent Application Publication
(10) Pub. No.: US 2013/0190738 A1
(43) Pub. Date: Jul. 25, 2013

(54) Laser system for the treatment of body tissue

(71) Applicants: Matjaz Lukac, Ljubljana (SI); Marko Kazic, Dob (SI)

(72) Inventors: Matjaz Lukac, Ljubljana (SI); Marko Kazic, Dob (SI)

(73) Assignee: FOTONA D.D., Ljubljana (SI)

(21) Appl. No.: 13/823,967

(22) PCT Filed: Sep. 24, 2010
(86) PCT No.: PCT/EP10/05853

§ 371 Date: Mar. 15, 2013

(51) Int. Cl. A61B 18/22

(52) U.S. Cl. CPC A61B 18/22
USPC 606/10

(57) ABSTRACT

The invention relates to a laser system for the treatment of body tissue (1) on an inner circumferential tissue surface (2). The laser system comprises a laser source (1) for the generation of a laser beam (3) and a handpiece (4) with a treatment head (5). The treatment head (5) extends along a longitudinal axis (6) and is adapted in an manner, that the longitudinal axis (6) of the treatment head (5) during operation is at least approximately parallel to the inner circumferential tissue surface (2). During operation the laser beam (3) enters the treatment head (5) in the direction of the longitudinal axis (6). A deflection mirror (7) is disposed in the treatment head (5) and guides the laser beam (3) radially outwards out of the treatment head (5) onto the inner circumferential tissue surface (2). Movable deflection means (8) for the laser beam (3) are provided to scan the inner circumferential tissue surface (2) within a treatment area (9) at least in a circumferential direction.

The invention relates to a device for the treatment of body tissue on an inner circumferential body surface.

Various laser wavelengths, such as Er:YAG (2.94 μm wavelength), Er,Cr:YSGG (2.73 μm wavelength), CO₂ (8-11 μm wavelength) have been advocated as possible and promising alternatives to conventional instruments in different medical and surgical disciplines. Due to their characteristic absorption maxima and thermal absorption coefficients, laser systems are considered to be suitable not only for the treatment of soft tissue structures but also for mineralized hard tissues. Contact-free laser ablation offers the opportunity of cutting bone and other hard tissue without friction that may cause additional thermal and mechanical trauma. Consequently the risk of cell death and delayed healing may be minimized. Furthermore, in contrast to conventional procedures no tissue particles debris is left on the tissue surface leading to a smear layer on the treated surface. This is due to the laser tissue ablation mechanism. Absorption and the following transformation of laser irradiation into heat results in a rapid phase change which, in turn, creates internal pressures, causing micro-fracturing and micro-explosive removal of the mineral phase of the hard tissues. Besides, during the laser ablation of the tissue, the vaporization of water leads to a fast removal of the tissue layers. The result is extremely clean and micro-structured tissue surface without thermal damage and smear layer, resulting in the reduced inflammatory response and accelerated tissue regeneration and attachment. This is for example important in implantology where faster attachment of the bone to the inserted implants is crucial for faster patient recovery time.

Nevertheless, drawbacks of laser hard tissue surgery such as a considerable amount of time needed, a missing depth control and highly sophisticated handling requirements are still formidable. An advantage of mechanical tools such as drills and saws is that the surgeon has a very good tactile contact with the treated tissue providing feedback to the surgeon regarding the speed of the procedure and the depth of the drilled hole or cut. For this reason laser bone cutting is still assessed to be inferior to many conventional as well as other methods, such as piezoelectric osteotomy.

In particular, when creating a hole in hard body tissue like bone material, mechanical tools are commonly still preferred. This preference however leaves open the handling of the a.m. issues like e.g. residual smear layers on the inner circumferential body tissue surface of said hole. The treatment results of the inner circumferential body tissue surface by mechanical means are unsatisfactory.

Similarly, it is sometimes desirable to use laser light to treat soft or hard tissues on difficult to reach inner circumferential body surfaces. Treatments may involve skin ablation, incision, excision, vaporization, coagulation, tightening, hemostasis or disinfection, and can be performed for example in vaginal, urinal, rectal, ENT (ear, nose and throat) and other procedures. The choice of the laser wavelength depends on a procedure and desired effect on the tissue, and is not limited to the hard tissue wavelengths mentioned above. For example, when hemostasis is desired, the surgeon may decide to use Nd:YAG (1.064 μm wavelength) or KTP:YAG (0.532 μm wavelength).

The invention has the object to provide means for treating body tissue on an inner circumferential body tissue surface, which are able to improve or modify the surface tissue, thereby giving tactile contact and feedback to the surgeon.

This object is solved by a laser system with the features of claim 1.

A laser system for the treatment of body tissue on an inner circumferential tissue surface is proposed, comprising a laser source for the generation of a laser beam and a handpiece with a treatment head. The treatment head extends along a longitudinal axis. The treatment head is adapted in a manner, that the longitudinal axis of the treatment head during operation is at least approximately parallel to the inner circumferential tissue surface. During operation the laser beam enters the treatment head in the direction of the longitudinal axis. A deflection mirror is disposed in the treatment head and guides the laser beam radially outwards out of the treatment head onto the inner circumferential tissue surface. Movable deflection means for the laser beam are provided, said movable deflection means being provided to scan the inner circumferential tissue surface within a treatment area at least in a circumferential direction.

The inventive device allows for an access to tissue surfaces, which are not easily accessible; and which exist in the inner side of a hole or a body opening. The slim treatment head of the handpiece may be axially inserted into the hole or into the body opening, thereby contacting the inner circumferential tissue surface. Despite of the cramped spatial conditions the deflection mirror provides, supported by the contacting of the treatment head with the inner circumferential tissue surface, that the focus of the laser beam is projected with high fluency on the inner circumferential tissue surface, thereby providing the desired tissue treatment. By means of the movable deflection means a predetermined scanning pattern may be scanned, which leads to the desired treatment result.

The inventive laser system is in particular suitable fort the post treatment of drilling holes in hard bone material e.g. in implantology. At first, the drilling hole is mechanically produced as usual. Subsequently the laser system is used as a laser grater or laser rasp, by means of which residual smear layers of loose bone material are cleared from the inner circumferential tissue surface of the drilling hole. In addition, the drilling hole may be brought to its nominal measure with improved precision. Finally, it is possible to achieve a desired surface quality and even a desired surface structuring. In addition to the treatment of hard bone material the inventive laser system is suitable for the treatment of soft body tissue like skin or the like, in particular for vaginal, urinal, rectal or ENT (ear, nose and throat) treatments.

In a preferred embodiment of the invention the deflection means comprise a rotation device for rotating the deflection minor about the longitudinal axis of the treatment head. In an advantageous embodiment the deflection mirror is rotationally driveable together with the treatment head about its longitudinal axis. The deflection minor is preferably flat.

With simple mechanical means and without intensive control effort the rotational movement of the mirror in particular together with the treatment head about its longitudinal axis can be realised. In connection with a flat deflection mirror no particular optical auxiliary means are required in order to project the focus of the laser beam onto the inner circumferential tissue surface. As a result of the rotational movement the inner circumferential tissue surface is scanned in the circumferential direction, leading to an evenly spread laser treatment. Therein it may be expedient to provide a manual positioning of the hand piece in the axial direction, which is easily possible due to the good tactile feedback to the surgeon. Thereby a complex scanner with a tilting mirror including its control device can be omitted. The device is simple and cost effective.

In a further preferred improvement a scanner for the laser beam is disposed on the input side of the treatment head and is adapted in a manner, that the treatment area is scanned by the laser beam parallel to the longitudinal axis of the treatment head.

By coordination of the scanner control and the control of the rotational deflection minor movement an arbitrary scanning pattern can be generated and adapted to the desired treatment task. A manually performed axial feed of the hand piece is not or only to a limited extent required. The surgeon needs only to position the hand piece at the right location, while the entire scanning pattern is scanned by the interaction of the rotating deflection mirror and the scanner.

In an alternative advantageous embodiment the deflection means comprise a scanner being movable about two axes, wherein the deflection mirror has a conical shape and is disposed with its apex facing the scanner.

According to this variant of the invention a mechanically rotated deflection mirror or a mechanically rotated treatment head is not required. By means of the scanner which is movable about two axes the laser beam is guided over the conical reflection surface of the conical deflection mirror in such a manner, that the inner circumferential tissue surface is scanned both in the circumferential direction and in the axial direction, thereby following a certain, without limitations creatable scanning pattern. Within the treatment head no mechanically movable parts are present. The treatment head itself does not perform any mechanically driven movement either, as a consequence of which the inner circumferential tissue surface does not have any contact to mechanically moved parts. The treatment head may be designed slim with low construction volume. This allows the treatment of even very small drill holes or openings. By means of a suitable scanner control device for the two axial scanner, even complex scanning patterns may be achieved. Such scanning patterns do not necessarily need to be evenly distributed in the circumferential direction. Referring to the circumferential direction certain angular sections may be excluded from the laser treatment.

It can be expedient that the deflection mirror is fixed to the treatment head by means of a carrier, wherein the carrier comprises carrier arms and windows between the carrier arms for the emerging laser beam.

This provides mechanically simple means for a secure fixing of the deflection mirror to the treatment head. The laser beam exits from the treatment head through said window onto the treatment area without any optical disturbance. The carrier arms may be designed sufficiently thin, according to which their shadowing effect is negligible.

In the alternative it may be preferred that the deflection mirror is fixed to the treatment head by means of a carrier, wherein the carrier is transparent and closed in the circumferential direction.

The transparent carrier may be manufactured of optical glass or other suitable material being transparent fort the laser beam. The laser beam may exit the treatment head in any desired angle referred to the circumferential direction without any shadowing effect. Besides carrying the deflection mirror, the transparent carrier acts as a protection device for the deflection mirror and the inner space of the treatment head, thereby preventing any pollution by body tissue debris.

In a preferred embodiment the movable deflection means comprise a control device, the control device being adapted in a manner, that the treatment area is scanned by the laser beam on circles with centres on the treatment area, wherein the laser beam is subjected to a circular feed about the longitudinal axis of the treatment head.

Alternatively it may be expedient that the deflection means comprise a control device, the control device being adapted in a manner, that the treatment area is scanned by the laser beam parallel to the longitudinal axis of the treatment head, wherein the laser beam is subjected to a circular feed about the longitudinal axis of the treatment head.

In a further alternative it may be preferable that the deflection means comprise a control device, the control device being adapted in a manner, that the treatment area is scanned by the laser beam in a random pattern, wherein the laser beam is subjected to a circular feed about the longitudinal axis of the treatment head.

A random pattern, combined with a circular feed about the longitudinal axis of the treatment head will result in a more homogenous treatment of the circumferential tissue surface. The homogeneity may be further improved by slight longitudinal and/or rotational movements of the handpiece.

In a still further alternative it may be preferable that the deflection means comprise a control device, the control device being adapted in a manner, that the treatment area is scanned by the laser beam solely in a circular way about the longitudinal axis of the treatment head.

The latter alternative leads to a reduction to a circular scanning pattern being scanned in a circular way about the longitudinal axis of the treatment head. As a consequence the control effort for the scanner can be minimized. In connection with a rotating deflection mirror an additional scanner may be entirely omitted. By providing a laser beam profile with a sufficient large diameter and preferable with a top hat shaped beam profile, the treatment area is evenly and homogenously illuminated without need for manual corrections.

Embodiments of the invention will be explained in the following with the aid of the drawing in more detail. It is shown in:

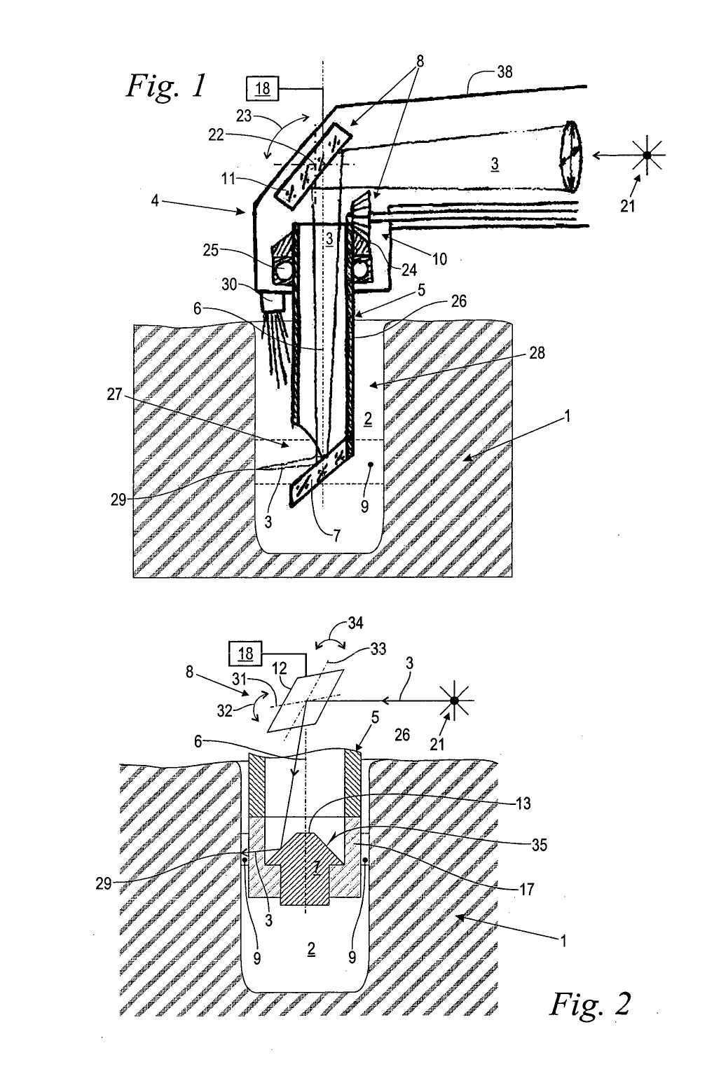

FIG. 1 in a schematic sectional view a first embodiment of an inventive laser system with a rotationally drivable treatment head carrying a deflection mirror;

FIG. 2 in a schematic sectional view a second embodiment of an inventive laser system with deflection means comprising a scanner being movable about two axes and a deflection mirror having a conical shape, with the deflection mirror being fixed to the treatment head by a transparent carrier;

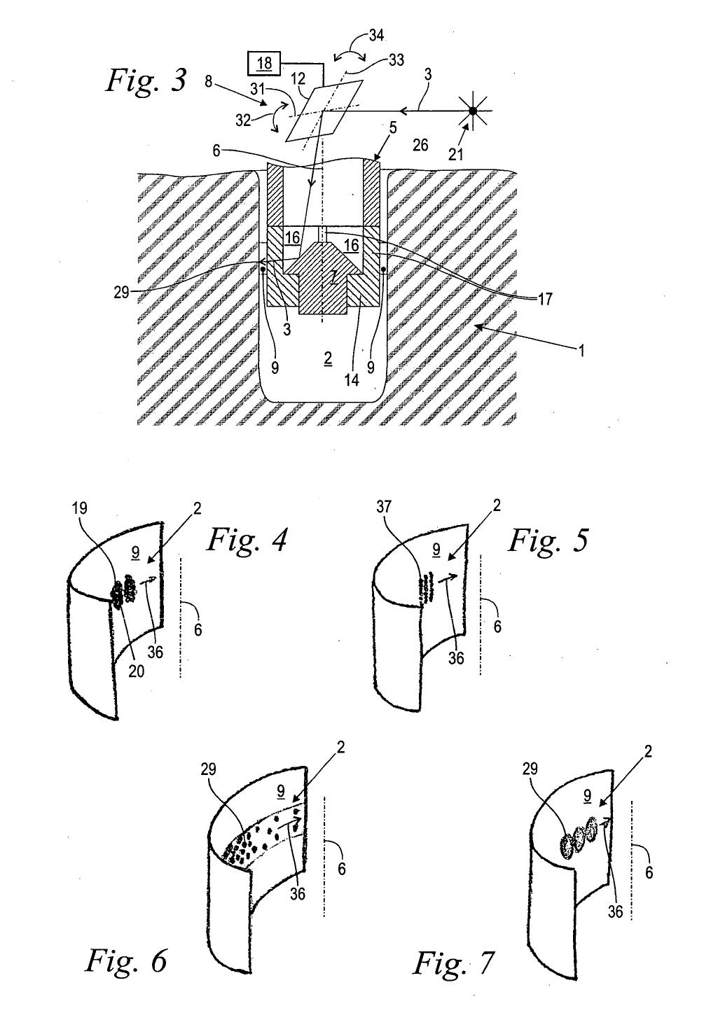

FIG. 3 a variant of the system according to FIG. 2 as a third embodiment with the carrier comprising carrier arms and windows;

FIG. 4 in a schematic perspective view a scanning pattern on the inner circumferential body surface in circles being combined with a circular feed about the longitudinal axis;

FIG. 5 a variant of the scanning pattern according to FIG. 4 scanning parallel to the longitudinal axis combined with a circular feed about the longitudinal axis;

FIG. 6 a further variant of the scanning pattern according to FIGS. 4 and 5 scanning a random pattern combined with a circular feed about the longitudinal axis;

FIG. 7 a further scanning pattern only in a circular way about the longitudinal axis of the treatment head.

FIG. 1 shows in a schematic sectional view a first example of the inventive laser system. The laser system comprises a handpiece 4 and a laser source 21 for generation of a laser beam 3. The laser source 21 is an Er:YAG-laser with a wave length of 2.94 μm, but may alternatively be, depending on the treated surface of body tissue 1 an Er,Cr:YSGG-laser (2.73 μm wave length) or a CO₂-laser (8-11 μm wave length) or a laser with any other wavelength. The laser beam 3 emitted from the laser source 21 may be guided into the hand piece 4 by means of an articulated arm or an optical fibre. It may also be expedient to place a laser source 21 in the handpiece 4.

In the shown embodiment the handpiece 4 comprises a base body 38 and a treatment head 5. The treatment head 5 extends along a longitudinal axis 6. A flat deflection minor 7 is disposed in the treatment head 5. The area of the deflection minor 7 is disposed in a 45° angle to the longitudinal axis 6. The treatment head 5 further comprises a tube 26, to which the deflection mirror 7 is fixedly connected. The deflection mirror 7 comprises an exit side, at which an opening 27 for the emerging laser beam 3 is provided in the tube 26. The opening 27 may be covered or closed by a transparent window.

The longitudinal axis 6 of the treatment head 5 may be disposed parallel or coaxial to the longitudinal axis of the base body 38. In the shown embodiment the longitudinal axis 6 is disposed in an angle thereto, said angle being at least approximately 90° as shown. However, different angles may be chosen as well. In order to deflect the laser beam 3 after entering the hand piece 4, an additional deflection mirror is disposed in the base body 38. Said additional deflection minor introduces the laser beam 3 into the treatment head 5 at least approximately parallel to the longitudinal axis 6. In addition, said additional deflection mirror forms a scanner 11 having a schematically shown control device 18. Under the control of the control device 18 the minor of the scanner 11 is rotationally movable about a rotational axis 22 according to an arrow 23. The rotational axis 22 is disposed perpendicular to the axis of the incoming laser beam 3 and to the axis of the laser beam portion being reflected from the scanner 11.

As shown in FIG. 1 the laser beam 3 entering the hand piece 4 has a certain diameter, from which the laser beam 3 is focussed along its path thorough the hand piece 4 onto an impingement point 29. By means of a suitable, not shown focussing optical arrangement the laser beam 3 may have different diameters at the impingement point 29. By adaption of said diameter and power of the laser source 29 the fluence in the impingement point 29 may be set to a desired value, thereby achieving the desired treatment result.

The inventive laser system comprises movable deflection means 8 for the laser beam 3. In the shown example, wherein the additional deflection mirror is embodied as a scanner 11, said upper scanner 11 is part of the movable deflection means 8. The movable deflection means 8 additionally comprise a rotation device 10 for rotating the lower deflection mirror 7 about the longitudinal axis 6 of the treatment head 5. It may be expedient to rotate the deflection mirror 7 alone, while the further parts of the treatment head 5 remain stationary. In the shown embodiment the entire treatment head 5 including the deflection mirror 7 is rotatably mounted at the base body 38 by means of a bearing 25, thereby being rotatable about the longitudinal axis 6. For rotatably driving the treatment head the rotation device 10 is provided with an angular gear 24, which is embodied as a miter gear. By driving the rotation device 10 e.g. by means of an electric motor, the deflection mirror 7, respectively the entire treatment head 5 is rotated about the longitudinal axis 6. The control device 18 is additionally adapted and provided for controlling the rotation device 10 and for providing a controlled coordination between the scanner 11 and the rotation device 10.

The inventive laser system is provided for treating body tissue 1 at an inner circumferential tissue surface 2. As an example for the body tissue 1 bone material is shown, in which beforehand a hole 28 was mechanically drilled. The hole 28 comprises an inner body tissue surface 2 circumferentially extending about the longitudinal axis of the hole 28. In operation the treatment head 5 is inserted in the hole 28 axially parallel to its axis, as a consequence of which the longitudinal axis 6 of the treatment head 5 is disposed at least approximately parallel to the inner circumferential tissue surface 2. The laser beam 3 being reflected from the scanner 11 enters the treatment head 5 at least approximately in the direction of the longitudinal axis 6. Afterwards, the laser beam 3 is deflected radially outwards by the deflection mirror 7, which is disposed in said 45° angle referred to the longitudinal axis 6. After being reflected at the deflection mirror 7 the laser beam 3 radially emerges from the treatment head 5 in an outward direction and impinges on the inner circumferential body surface 2 on the impingement point 29.

By rotating the deflection mirror 7, respectively the entire treatment head 5 the impingement point 29 is moved within the treatment area 9 of the inner circumferential tissue surface 2 in the circumferential direction both about the longitudinal axis 6 of the treatment head 5 and about the longitudinal axis of the hole 28. Hereby the treatment area 9 is scanned by the laser beam 3 in the circumferential direction. As a consequence of an additional movement of the scanner 11 about its rotational axis 22 according to the arrow 23 the treatment area 9 is additionally scanned by the laser beam 3 parallel to the longitudinal axis 6 of the treatment head 5 and to the longitudinal axis of the hole 28 respectively. By combining scanning movements both in axial and in circumferential direction different desired scanning patterns can be achieved, as exemplarily shown in and described infra related to FIGS. 4 to 7.

For cooling and rinsing the treatment area 9 a spray device 30 is provided at the hand piece 4, by means of which air and/or water or other suitable media may be supplied to the treatment area 9, if desired.

FIG. 2 shows in a schematic sectional view a variant of the arrangement according to FIG. 1 as a further example of the invention, wherein the base body 38 of FIG. 1 is not shown for the sake of simplicity. No rotation device 10 (FIG. 1) is provided in the embodiment of FIG. 2. Instead the movable deflection means 8 comprise a scanner 12 in form of a deflection mirror, which is rotationally movable about two perpendicularly to each other disposed rotational axes 31, 33 according to arrows 32, 34. Like the scanner 11 of FIG. 1 the scanner 12 of FIG. 2 is controlled by the control device 18.

The deflection mirror 7 is embodied as a cone comprising a conical mirror surface 35 and an apex 13. The central axis of the conical deflection mirror 7 is disposed coaxially to the longitudinal axis 6 of the treatment head 5, wherein the apex 13 is facing the scanner 12. The aperture angle of the conical mirror surface 35 is at least approximately 90°, but may have a different value. The conical deflection mirror 7 is embodied as a metal body having a reflective, polished metal mirror surface 35. In lieu of the polished mirror surface 35 a reflective coating may be provided.

As a consequence of said aperture angle the incoming laser beam 6 is radial outwardly reflected by the conical mirror surface 35 and impinges on the treatment area 9 of the inner circumferential tissue surface 2 at the impingement point 29. By rotationally moving the scanner 12 about its two rotational axes 31, 33 any point of the conical mirror surface 31 may be scanned referred to both the circumferential and radial direction. Thereby the radially emerging laser beam 3 may reach with its impingement point 29 any location on the treatment area 9 referred to both the circumferential direction and the axial direction, the latter being predetermined by the longitudinal axis 6.

The conical deflection mirror 7 is fixedly attached to the tube 26 of the treatment head 5 by means of a transparent carrier 17. The transparent carrier 17 may be made of optical glass and is entirely closed in the circumferential direction about the longitudinal axis 6. Hereby the transparent carrier 17 does not only act as a carrier for the deflection mirror 7, but also acts as a protective window for the inner space of the treatment head 5, in particular including the reflective conical mirror surface 35. In addition, the laser beam 3 can unobstructedly transit the transparent carrier 17 radially outwards to the treatment area 9.

FIG. 3 shows a variant of the arrangement according to FIG. 2, wherein as a replacement of the transparent carrier 17 (FIG. 2) a carrier 14 is provided for carrying and fixing the deflection mirror 7 to the tube 26. The carrier 14 comprises carrier arms 15 for fixing the arrangement to the tube 26, wherein the carrier arms 15 are disposed parallel to the longitudinal axis 6. Windows 16 are provided between the carrier arms 15, through which the reflected laser beam 3 emerges to the treatment area 9. The windows 16 are open, but may also be embodied as transparent protective windows of glass or any other suitable material being transparent for the laser beam 3, and having a function being comparable to the transparent carrier 17 of FIG. 2.

With respect to all other described and/or shown features and reference signs, and if not otherwise stated, the arrangements of FIGS. 2 and 3 concur with each other and with the arrangement of FIG. 1.

The inventive laser system including its control device 18 is adapted to the operated in an inventive method as follows: In general, the treatment area 9 of the inner circumferential body surface 2 is scanned in particular patterns. Said scanning is performed to remove residual smear layers from body tissue 1 being e.g. hard bone material, in which holes 28 (FIGS. 1 to 3) were mechanically drilled beforehand. In addition, a certain surface structure of the inner circumferential tissue surface and/or a correction of the hole profile may be achieved. It is further possible to use the inventive arrangement and method for treatment of soft body tissue such as skin or the like in surgical cuts or existing body openings, as may be desired e.g. along with vaginal treatments. Related preferred scanning patterns are schematically depicted in FIGS. 4 to 7, wherein sections of the inner circumferential tissue surface 2 are perspectively shown. As in FIGS. 1 to 3 the longitudinal axis 6 of the treatment head 5 is disposed at least approximately parallel to the circumferential tissue surface 2 and its treatment area 9.

For certain treatment tasks sufficiently high fluencies of the laser beam 3 are required. In such cases the laser beam 3 is focussed on the impingement point 29 with a small, nearly pinpoint shaped diameter, wherein said diameter is small compared to the axial extension of the treatment area 9. This requires a scanning of the treatment area 9 both in axial and circumferential direction, as shown in FIGS. 4 to 6.

According to the example of FIG. 4 the treatment area 9 of the inner circumferential tissue surface 2 is scanned in a pattern, in which the impingement point 29 (FIGS. 1 to 3) is guided on circles 19, wherein the circles 19 including their centers 20 are disposed on the treatment area 9. The circles 19 may be scanned along their circumference or across their entire circular area. In addition to said circular scanning about the centers 20 a circular feed of the impingement point 29 about the longitudinal axis 6 according to an arrow 36 is provided, in consequence of which the treatment area 9 is entirely scanned both in circumferential and axial direction related to the longitudinal axis 6.

In the embodiment according to FIG. 5 lines 37 are provided instead of circles 19 (FIG. 4), said lines 37 being disposed parallel to the longitudinal axis 6, and along which the treatment area 9 is scanned. In addition thereto a circular feed of the impingement point 29 about the longitudinal axis in the direction of the arrow 36 is provided.

A further embodiment is shown in FIG. 6, according to which the treatment area 9 is scanned in a random pattern. The impingement points 29 are randomly spread over the treatment area 9. Again an additional feed in the circumferential direction about the longitudinal axis 6 according to the arrow 36 is provided.

In the embodiments according to FIGS. 4 to 6 two separate scanning patterns are provided. The first scanning pattern is either circular (FIG. 4), linear (FIG. 5) or random (FIG. 6). The second scanning pattern is circular about the longitudinal axis 6. It may be expedient to switch between both types of patterns back and forth in order to scan the entire treatment area 9. As an alternative, it may be expedient to continuously superpose both scanning patterns for scanning the entire treatment area 9.

In a further variant of the invention, as schematically shown in FIG. 7, an axial scanning of the treatment area 9 may be omitted. This is in particular suitable for the case, wherein instead of nearly pinpoint shaped impingement points 29 (FIGS. 4 to 6) planar impingement points 29 of larger diameter are projected onto the treatment area 9, which results in an axial extension of the scanned treatment area 9 without axial scanning movement components. Herein this treatment area 9 is solely scanned in a circular manner about the longitudinal axis 6 according to the arrow 36. In the embodiments according to FIGS. 2 and 3 this may be achieved by a suitable control of the scanner 12 and by a circular scanning of the conical deflection mirror 7. In the embodiment according to FIG. 1 said circular scanning pattern is achieved by the rotational movement of the treatment head 5 without any further measures. Only the rotation device 10 and the rotational movement of deflection mirror 7 is required as movable deflection means 8, while the scanner 11 may be omitted.

What is claimed is:

1.-12. (canceled)

13. A laser system for the treatment of body tissue (21) on an inner circumferential tissue surface (2), the laser system comprising:
a laser source (1) generating a laser beam (3);
a handpiece (4) comprising a treatment head (5), wherein the treatment head (5) has a longitudinal axis (6) and wherein the treatment head (5) is configured such that the longitudinal axis (6) of the treatment head (5) during operation is at least approximately parallel to an inner circumferential tissue surface (2);
wherein during operation the laser beam (3) enters the treatment head (5) in a direction of the longitudinal axis (6);
a deflection mirror (7) disposed in the treatment head (5) and guiding the laser beam (3) in a radial direction outwardly out of the treatment head (5) onto the inner circumferential tissue surface (2);
movable deflection means (8) for the laser beam (3) comprising a scanner (12) movable about two axes;
wherein the deflection mirror (7) has a conical shape and is disposed with an apex (3) thereof so as to face the scanner (12);
wherein the scanner (12) is controlled for at least a circular scanning of the conical deflection mirror (7) so as to scan the inner circumferential tissue surface (2) within a treatment area (9) at least in a circumferential direction.

14. The laser system according to claim 13, comprising a carrier (14), wherein the deflection mirror (7) is fixed to the treatment head (5) by the carrier (14), wherein the carrier (14) comprises carrier arms (15) and windows (16) between the carrier arms (15) for the laser beam (3).

15. The laser system according to claim 13, comprising a carrier (17), wherein the deflection mirror (7) is fixed to the treatment head (5) by the carrier (17), wherein the carrier (17) is transparent and closed in circumferential direction.

16. The laser system according to claim 13, wherein the deflection means (8) comprise a control device (18), the control device (18) being adapted to scan the treatment area (9) with the laser beam (3) along circles (19) with centers (20) located on the treatment area (9), wherein the laser beam (3) is subjected to a circular feed action about the longitudinal axis of the treatment head (5).

17. The laser system according to claim 13, wherein the deflection means (8) comprise a control device (18), the control device (18) being adapted to scan the treatment area (9) with the laser beam (3) parallel to the longitudinal axis of the treatment head (5), wherein the laser beam (3) is subjected to a circular feed action about the longitudinal axis of the treatment head (5).

18. The laser system according to claim 13, wherein the deflection means (8) comprise a control device (18), the control device (18) being adapted to scan the treatment area (9) with the laser beam (3) in a random pattern, wherein the laser beam (3) is subjected to a circular feed action about the longitudinal axis of the treatment head (5).

19. The laser system according to claim 13, wherein the deflection means (8) comprise a control device (18), the control device (18) being adapted to scan the treatment area (9) with the laser beam (3) solely in a circular pattern about the longitudinal axis of the treatment head (5).

20. A method for the treatment of body tissue (21) on an inner circumferential tissue surface (2) by a laser system, wherein the laser system comprises a laser source (1) and a handpiece (4) with a treatment head (5), wherein the treatment head (5) has a longitudinal axis (6), wherein movable deflection means (8) for the laser beam (3) are provided comprising a scanner (12) being movable about two axes, wherein a deflection mirror (7) is disposed in the treatment head (5), wherein the deflection mirror (7) has a conical shape and is disposed with an apex (13) thereof so as to face the scanner (12); the method comprising:
positioning the treatment head such that the longitudinal axis (6) of the treatment head (5) is at least approximately parallel to the inner circumferential tissue surface (2),
generating a laser beam (3) by the laser source (1) and causing the laser beam (3) to enter into the treatment head (5) in a direction of the longitudinal axis (6),
controlling the scanner (12) such that at least a circular scanning of the conical deflection mirror (7) with the laser beam (3) is carried out;
guiding the laser beam (3) in radial direction outwardly out of the treatment head (5) onto the inner circumferential tissue surface (2); and
scanning the inner circumferential tissue surface (2) with the laser beam (3) within a treatment area (9) at least in a circumferential direction.

21. The method according to claim 20, wherein in the step of scanning the laser beam (3) is guided on circles (19) with centres (20) of the circles located on the treatment area (9) and the laser beam (3) is subjected to a circular feed action about the longitudinal axis of the treatment head (5).

22. The method according to claim 20, wherein in the step of scanning the laser beam (3) is guided parallel to the longitudinal axis of the treatment head (5) and the laser beam (3) is subjected to a circular feed action about the longitudinal axis of the treatment head (5).

23. The method according to claim 20, wherein in the step of scanning the laser beam (3) is guided in a random pattern and the laser beam (3) is subjected to a circular feed action about the longitudinal axis of the treatment head (5).

24. The method according to claim 20, wherein in the step of scanning the laser beam (3) is guided solely in a circular way about the longitudinal axis of the treatment head (5).

25. The method according to claim 20, wherein in the step of scanning the laser beam (3) is guided such that sections of the inner circumferential tissue surface (2) are excluded from the laser treatment.

26. The method according to claim 20, wherein the body tissue (1) is hard bone material, further comprising the step of drilling a hole into the hard bone material to produce the circumferential tissue surface (2) to be treated so that the treatment area (9) is located on the circumferential wall of the hole (28).

27. The method according to claim 26, further comprising the step of removing residual smear layers from the hard bone material by scanning the inner circumferential tissue surface (2) of the hole (28) with the laser beam (3).

28. The method according to claim 26, further comprising the step of creating a surface structure of the inner circumferential tissue surface and the step of correcting a hole profile of the hole by scanning the inner circumferential tissue surface (2) of the hole (28) with the laser beam (3).

29. The method according to claim 26, further comprising the step of creating a surface structure of the inner circumferential tissue surface by scanning the inner circumferential tissue surface (2) of the hole (28) with the laser beam (3).

30. The method according to claim 26, further comprising the step of correcting a hole profile of the hole by scanning the inner circumferential tissue surface (2) of the hole (28) with the laser beam (3).

31. The method according to claim 20, wherein the circumferential tissue surface (2) to be treated is a vaginal wall and wherein the treatment area (9) is located on the circumferential vaginal wall.
